(19) United States
(12) Patent Application Publication
(10) Pub. No.: US 2023/0115691 A1
(43) Pub. Date: Apr. 13, 2023

(54) COMPUTER-IMPLEMENTED METHOD TO GATHER AND STORE EPIDEMIOLOGICAL DATA TO CALCULATE DAMAGES PAYMENTS OF AN INFECTIOUS DISEASE SPREADERS FUND

(71) Applicant: Levan Roberto Djaparidze, Buenos Aires (AR)

(72) Inventor: Levan Roberto Djaparidze, Buenos Aires (AR)

(21) Appl. No.: 17/661,793

(22) Filed: May 3, 2022

Related U.S. Application Data
(60) Provisional application No. 63/254,246, filed on Oct. 11, 2021.

(51) Int. Cl. G06Q 40/08; G16H 50/80; G16H 70/60

(52) U.S. Cl. CPC G06Q 40/08; G16H 50/80; G16H 70/60

(57) ABSTRACT

Computer-implemented method for calculating disease damage payments due to spreaders fault associated to an isolation exemption insurance policy (IEIP). Based upon an assessment of expected disease damages and the share fault of injured and spreaders for not isolating, insurer companies agree a method to computationally gather and store epidemiological data to calculate payments made to and by the Spreaders fund to cover the portion of disease damages suffered by the injured that are fault of spreaders. Whenever a location suffers an infectious disease epidemic with significant presymptomatic contagions, and mandatory isolations are imposed to asymptomatic individuals to reduce spreading, such individuals might be offered an IEIP to avoid those mandatory isolations. The computer-implemented method can be updated over time. The portion of disease damages that is fault of the injured is covered by each insurer.

### CROSS-REFERENCE TO RELATED APPLICATIONS

This application claims the benefit of priority to U.S. Provisional Application No. 63/254,246 filed Oct. 11, 2021, the contents of which are incorporated herein by reference.

### FIELD OF THE INVENTION

The present disclosure relates to insurance, and more specifically, to systems and methods for calculating payments when injured and spreaders of infectious-contagious diseases share fault.

### BACKGROUND OF THE INVENTION

The SARS-CoV-2 virus is simultaneously: airborne highly contagious, produces a disease (covid19) severe enough to overwhelm health systems, has a short infectiousness interval that can be asymptomatic (pre-symptomatic, paucisymptomatic, or mild-symptomatic) (3)(4)(8), and hence classic Test/Trace/Isolate strategies are useless (i.e. infected do not report symptoms), or cannot be applied alone (i.e. imagine contagions in public night clubs), or it is impractical to determine individual infectiousness (e.g. daily PCR testing), and so the virus spreads without being traced.

In face of this challenge governments imposed mandatory isolations (e.g. work/meeting/circulation restrictions) also known as Non-Pharmaceutical Interventions (NPIs). NPIs were strict enough to pause virus spreading growth, as verified in (2) for five locations in Europe where daily covid19 deaths curves peaked roughly 20 days after NPIs started.

SARS-CoV-2 spreading growth could also be paused using a more focused and voluntary approach (e.g. only among elderly) like in Stockholm (2), but nevertheless some (mandatory) NPIs were in place there too.

State mandatory restrictions to work, meet, or circulate might have violated individual rights and also caused colossal economic damages (10). Particularly for the SARS-CoV-2 epidemic, economic damages caused by this kind of response were at least an order of magnitude higher than quality-adjusted life year (QALY) disease damages: as July 2021 the USD had 631.915 deaths, with median 76.5 years old and +2 comorbidities (6), and the US GDP drop during 2020 was 3.5% (10) (i.e. USD 1.5 M per death).

What went wrong? The SARS-CoV-2 epidemic required complex risk assessment and governments are not the best equipped to do it (insurance companies are). The solution for this epidemic (and future similar ones) might be an isolation exemption insurance policy (IEIP).

Whenever NPIs are imposed during an epidemic, any individual willing to be exempt from complete isolation must take out insurance to cover disease damages to himself and also to third parties for spreading the virus (including years to live loss due to deaths or sequels and hospitalizations expenses). Why should spreaders pay an issue premium to cover covid19 disease-related damages of third parties they will never see? Because spreading a virus capable of producing disease causes damage.

Although an individual going out to work during an epidemic may cause damages to third parties (and to himself), if the cost of covering the damages on third parties (or his own hospitalization expenses) is lower than his personal perceived cost of isolating he might decide to go out anyway. Going out even though you can cause damage and covering the risk may at first seem heartless, but that is exactly what happens every time you drive your car or take a plane. Complete isolation of an elderly individual not willing to suffer damages from your behavior should include living inside a concrete bunker to avoid your car ending up inside his living room or the plane falling and destroying his roof.

Of course, local government bodies that are imposing mandatory isolations would have to make it legal to avoid them with insurance, but this by no means would be a novel behavior, since governments have already massively allowed “key personnel” to avoid isolations.

Intensive Care Units (ICU) scarcity at the beginning of the epidemic may enormously increase disease damages due to improper medical attention. But if allowed, an increase in ICU hospitalization prices fuels rapid ICU expansion, and that risk is eliminated. Most likely the insurance could have exclusions, for example going out if you are symptomatic or entering nursing homes or non-covid19 hospitals without proper safety measures.

The proposed insurance would also help individuals to assess the personal risk they are subject to (e.g. for different isolation brackets) by knowing the issue premium portion that covers self personal damages. At some point, a waiver to the personal damages claim will be the option for vulnerable individuals wanting to isolate below some threshold.

Injured are also spreaders because they are not completely isolated and they got infected, a consequence of this would be that no individual without insurance could claim covid19 disease damages. Anyway, governments would likely allow some uninsured isolation exemptions and cover the difference.

The disease damages caused by infections can be thought as the result of accidents, with fault shared between the injured and spreaders. The injured have some fault because if they would have been completely isolated, they wouldn’t have got infected, and the spreaders cannot bear the entire fault because they don’t have unequivocal symptoms and/or they are unaware of their infectiousness.

In an epidemic the damaging spreaders cannot be identified, like freeway insurance that pays damages for an accident caused by the rubber band from an anonymous broken tire, but unlike a freeway where the same insurer is covering both the injured and the anonymous guilty, injured and spreaders might have different insurers.

Although a single government funded insurer offering IEIP to all individuals would be an improvement over unavoidable NPIs, if disease damages are underestimated tax payers would be subsidizing spreaders, and if it is overestimated, spreaders would be unfairly penalized because they do not have another option.

Competition between insurers offering IEIPs, where risk and premium prices are a private bet balancing uncertainty and market share, is desirable.

Some state of the art insurance proposals tried to go beyond current health insurance but addressing the issue from a classical approach, therefore proposing cover the damages suffered by business and individuals due to government restrictions (15), and therefore, the covering of the damages suffered by business and individuals due to the government restrictions with this traditional approach do not propose, neither allow to deduce insurance for allowing individuals or business to be exempt of those restrictions by covering related disease damages, nor do they depict a system and much less any computer-implemented method capable of instrument it.

The isolation exemption insurance policy and its associated spreaders fund proposed here are disruptive innovations, since never before the insurance industry has proposed an insurance system balancing the rights of contagious-infectious disease spreaders to selectively avoid NPIs with the rights of injured to be compensated for the damages.

### SUMMARY OF THE INVENTION

The present inventions provide insurers with a system and method to calculate infectious disease damage payments due to spreaders fault. The disease damages, as used herein, means any damage caused by the disease such as years to live loss due to death or sequels, reduced quality of life or earning capacity, disability/physical limitations, and hospitalizations expenses, for example physical limitations due to post-COVID-19 lung fibrosis. The disease damage payment, as used herein, means any amount of money paid to compensate disease damages, for example the amount of money the spreaders fund pays to insurers on a given week regarding the disease damages of a vaccinated elderly male isolating at high level.

FIG. 1 summarizes participants and interactions. Insured individuals might be grouped in Risk groups (RG) according to: disease damage risk (e.g. age, comorbidities), isolation level, susceptibility to getting infected, infectiousness if infected, etc. A risk group, as used herein, means any group of individuals sharing the similar risks according to some characteristics such as disease damage risk (e.g. age, sex, blood type, comorbidities), isolation level, susceptibility to getting infected, infectiousness if infected, vaccination status, previous infection (recovered or not), for example vaccinated elderly males isolating at high level. The premiums paid for the IEIP (i.e. price to purchase insurance) for individuals that belong to the same RG might be different for each insurer.

All damages suffered by injured individuals might be assigned to the period of time (e.g. day, week, or month) in which they were infected, and the epidemiological data in that date might be gathered and stored for future calculations. Regarding such epidemiological data, with the objective of providing a further explanation to the reader, as used herein, it is addressed to any data related to the epidemic such as cases, disease and hospitalization records, deaths certificates, isolation levels within the same RG or with other RG for infecting or getting infected, amount of infectious and susceptible individuals of each RG, relative infectiousness and susceptibility, for example amount of cases on a given week of healthy <60 unvaccinated individuals not isolating at all.

Namely, the period of time, as used herein, means days, weeks, months, years, i.e. any kind of magnitude to measure time. To the effects of calculating damage fault due to spreaders, disease damage claims might be tabulated for each RG, for example in the same manner that with occupational hazard insurance. The disease damage claims, as used herein, means any amount of money requested by a policyholder to an insurance for compensation of covered loss, for example to cover physical limitations due to post-COVID-19 lung fibrosis. Nevertheless, disease damage claims paid to injured by their own insurer may differ from the tabulated ones.

When injured individuals of a RG, claim disease damages due to infections occurred during a period (most likely claims will take place after that period of infection), the Spreaders fund calculates the share of that claim that was fault of all RG not isolating in that period, and payments due to damage fault of spreaders flow from insurers to the Spreaders fund and then back to insurers and finally to the injured. Payments to the injured made by their insurer will also include the portion of the damages that was fault of the injured.

Future damage claims due to infections in some period are uncertain, the Spreaders fund payment calculation method agreed between insurers only depicts the share that will correspond to each RG of IEIP holders, most likely insurers (maybe with government intervention too) will agree the calculation method for a given period before it elapses, and each insurer will then include that calculation into their own actuarial methods to estimate the future amount of payments for that period to calculate the IEIP issue premiums it will charge to their own insured.

If the agreed method bears the entire fault to the spreaders, all payments are made by the Spreaders fund. If the entire fault is assigned to the injured (Spreaders fund payments = 0) then the IEIP reverts to a classic disease insurance policy and is not covered by this invention.

Once vaccinated or recovered from an infection, individuals might be removed from their previous RG and included into a new RG because they have lower susceptibility of being infected and/or infectious, and hence their share fault will be reduced, and issue premiums for these vaccinated and/or recovered RG will be lower than for unvaccinated.

Since there is a large proportion of asymptomatic infections (5), unvaccinated naïve (not recovered, unvaccinated without prior infections) individuals might regularly take antibody tests, and if positive, provide them as prove of being recovered and thus obtain lower IEIP issue premiums.

NPIs could be subtle, for example bars and concert halls being mandated by the NYC government not to accept unvaccinated individuals (13). IEIP could be offered to individuals wanting to avoid those NPIs or purchased on their behalf by restaurants wanting to avoid that restriction.

Fines to the unvaccinated like the ones recently proposed in Austria for covid19 (17), or ruled as constitutional in 1905 in Massachusetts for smallpox (18), are a step in the right direction since individuals can pay to be exempt of isolations (i.e. unvaccinated that paid the fine are allowed to attend public spaces), but they are inevitably unfair, because disease damages caused by the spreader can turn out to be higher/lower than the value of the fine, and hence governments would end up subsidizing/penalizing the spreader.

Oddly the emergence of a new variant can increase the susceptibility of being infected and/or infectious for vaccinated individuals, see in (16) negative vaccine efficacy (VE) to prevent symptomatic infections of Omicron (VE = -18% for PF:D2:14+ in Table 2). In that case, issue premiums will be higher for individuals vaccinated (e.g. after 141 days of the 2^{nd} dose) than for the unvaccinated naive!

One consequence of IEIP being available to avoid mandatory isolations is that damage claims to governments due to imposing them might be capped to the IEIP premium cost, because they could be avoided by purchasing an IEIP.

Furthermore, insurance companies might have an incentive to funnel funds into unbiased research in order to accurately calculate premiums, for example trying to determine as soon as possible Infection Fatality Rates, sequels, vaccine efficacy, masking efficacy, etc.

### BRIEF DESCRIPTION OF THE DRAWINGS

FIG. 1, shows a System and computer-implemented method to gather and store epidemiological data to calculate damage payments of an infectious disease spreaders fund. The Spreaders fund calculates payments due to share fault of spreaders and they flow from insurers (that previously collected premiums from insured not isolating) to the insurers that had damage claims from their injured insured;

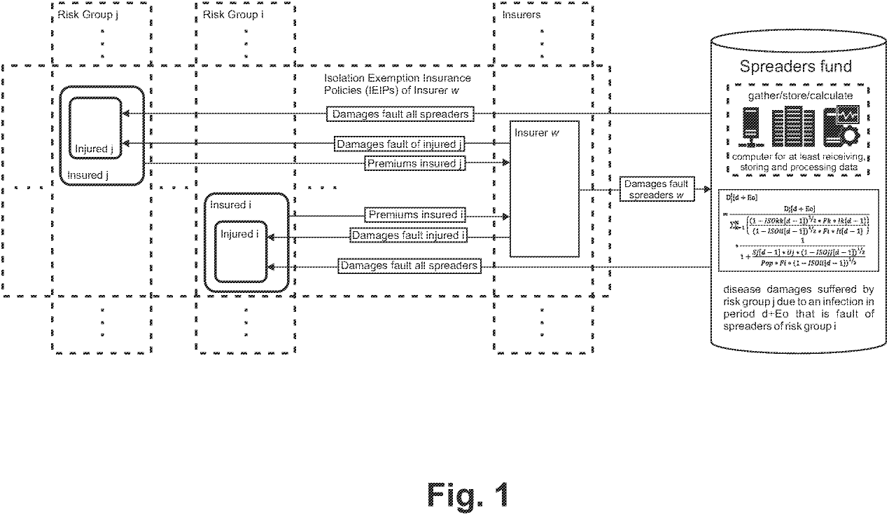

FIG. 2, shows an infectious disease basic epidemiological SEIR model, i.e. a Classic SEIR epidemiological model where individuals belong to either one of four compartments (Susceptible, Exposed, Infectious, Recovered);

FIG. 3, shows a 2-stratum SEIR model, i.e. an Extended 2-stratum SEIR model published in (1) that allows modeling stratified isolations for two groups: vulnerable and healthy <60;

FIG. 4 shows three scenarios for different isolation levels using real epidemic data from the Community of Madrid, with estimations of cumulative disease damage payments made by the Spreaders fund for healthy <60 and vulnerable IEIP holders; and

FIG. 5 shows a flow chart showing a computer-implemented method for calculating disease damage payments due to spreaders fault associated with an isolation exemption insurance policy (IEIP).

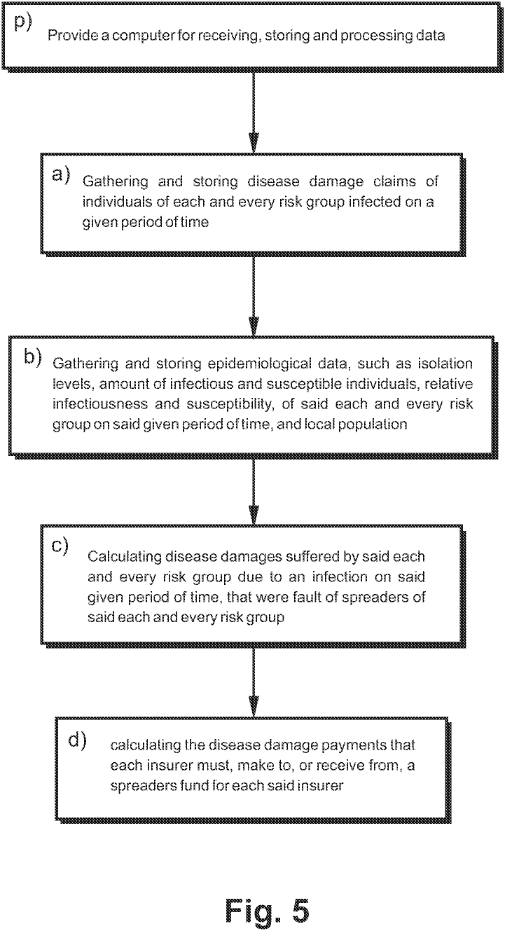

### DETAILED DESCRIPTION OF THE INVENTION

The present disclosure is directed to a computer-implemented method for calculating disease damage payments due to spreaders fault associated with an isolation exemption insurance policy (IEIP). For the purposes of the present invention, disease damage payment refers to the economic payment that each insurer must make to, or receive from, a spreaders fund for each said insurer.

For the purpose of explaining the kind of calculation used by the computer-implemented method of the present invention, it is further provided above an exemplary payment calculation method. A spreaders fault, as used herein, means the proportion of fault regarding the infections (i.e. accidents) that is assigned to the spreaders and not to the injured, for example the percentage of fault assigned to healthy <60 not isolating at all in the infections of elderly isolating at high level. Furthermore, a isolation exemption insurance policy (IEIP), as used herein, means document detailing the terms and conditions of a contract or of insurance including declarations, insuring agreements, definitions, exclusions, conditions and endorsements, including unilateral contracts by governments or other entities, for example a IEIP provided by governments to homeless.

Namely, since the method of the present invention requires the storage of information, and processing of such information in a dynamic and complex way, a person skilled in the art will well understand that its implementation by computer is essential. Like a skilled person will understand, the computer suitable for carrying out the computer-implemented method of the present invention includes the necessary physical devices for at least receiving, storing and processing data. Like a skilled person will understand, the physical devices may comprise, user interface devices, information gathering devices, information processor or central processing unit (CPU), storage devices, computer-readable [storage] medium, display devices, and the like. Therefore, in order to implement the method disclosed herein, a computer is provided.

Therefore, for the purposes of the present invention, since it is necessary to use a sequence of calculations and algorithms carried out by a computer, then it is necessary p) the provision of a computer for receiving, storing and processing data. Namely, a computer, as used herein, means an electronic device for storing and processing data, typically in binary form, according to instructions given to it in a variable program.

The computer-implemented method comprises the followings steps:
a) gathering and storing disease damage claims of individuals of each and every risk group infected on a given period of time,
b) gathering and storing epidemiological data, such as isolation levels, amount of infectious and susceptible individuals, relative infectiousness and susceptibility, of said each and every risk group on said given period of time, and local population,
c) calculating disease damages suffered by said each and every risk group due to an infection on said given period of time, that were fault of spreaders of said each and every risk group,
d) calculating the disease damage payments that each insurer must make to, or receive from, a spreaders fund for each said insurer.

In order to give more context to the above definitions in reference to the proposed method, an explanation of concepts is provided herein below as follows, which are merely clarifying and do not constitute new matter not already specified in steps a) to d) above.

The computer-implemented method starts performing the gathering and storing disease damage claims of individuals of each and every risk group infected on a given period of time; this is essential as it is the way in which the disease damage claims can be linked to infections occurred in a previous period of time, for example hospitalizations expenses due to intubation during the first for 3 weeks in March might be the result of an infection occurred during the 3rd week of February.

Following, the computer-implemented method performs the gathering and storing of epidemiological data, such as isolation levels, amount of infectious and susceptible individuals, relative infectiousness and susceptibility, of said each and every risk group on said given period of time, and local population; this is essential as it is the way in which disease damage payments can be calculated for said period of time, for example isolation levels of spreaders during the 3rd week of February might have been lower than during the first 3 weeks of March en hence spreaders fault regarding those disease damage claims is higher.

Specifically, the isolation levels refer to numerical values resulting from a set of restrictions (also known as Non-Pharmaceutical Interventions) and/or usual behavior that diminish interactions with other individuals (e.g. breathing same air) and that therefore lower the probability of contagions, for example the prohibition of gatherings of more than 50 people or the prohibition to enter pubs if not vaccinated. The amount of infectious and susceptible individuals refers to the estimated number of individuals being infectious and the number of individuals susceptible of becoming infectious when exposed to the pathogen, for example infectious individuals are known to be much higher than reported cases and susceptible individuals are known to be less than individuals without prior confirmed infection. The relative infectiousness and susceptibility refer to the comparative degree of infectiousness and susceptibility between different individuals, for example a higher proportion of young individuals develop asymptomatic infections compared with elderly individuals and hence they are relatively less infectious, and children are known to be less susceptible to be infected than the elderly. The local population as used herein means any limited territory where calculations are performed, for example local population in Paris might include the Paris City departments: 75, 92, 93, 94 and also Greater Paris 95, 78, 91, and 77 departments.

Following, the computer-implemented method performs the calculating disease damages suffered by said each and every risk group due to an infection on said given period of time, that were fault of spreaders of said each and every risk group; this is essential as it is the way in which the actual amount of payments can be calculated, since insurers might have agreed the method to calculate what proportion of damages must be paid by and to each RG, but the actual amount of money paid requires knowing the actual disease damages.

Finally, the computer-implemented method performs the calculating the disease damage payments that each insurer must make to, or receive from, a spreaders fund for each said insurer; this is essential as it is the way in which injured are compensated and insurers covering spreaders are charged the disease damages. Particularly, the calculated disease damage payments that each insurer must, make to, or receive from, a spreaders fund for each said insurer can be obtained and as exemplified below, using the results of formula {10} which depicts for any given pair of groups j and i (including j=i), the amount of disease damages suffered by RG j due to an infection in period d+Eo that is fault of spreaders of RG i. Formula {10} is deducted from a reasonable set of assumptions, gathered data and a particular SEIR epidemiological model, but any other set of assumptions and/or epidemiological model can be used to agree a calculation between insurers, for example a more sophisticated approach including estimations of ISOij (ij element in the contact matrix) using proximity with a mobile app, like “Exposure Notifications” from Google and Apple (7), and instead of using {8} to calculate the proportion of fault in the joint interaction, discriminate the right of each RG to be in each place where interactions happen (e.g. children not isolating from elderly in a primary schools might not have the same fault share that in restaurants).

Although a specific series of formulas have been indicated herein allowing the user to calculate the disease damage payments that each insurer must make to, or receive from, a spreaders fund for each said insurer, the computer-implemented method disclosed herein, may agreed other formulas and/or steps to calculate the disease damage payments. Said other formulas, as used herein, means any formula different than {10} agreed between insurers (or governments or other entities) to calculate disease damage payments made by the spreaders fund, for example simply dividing all disease damages by the total number of individuals in the local population.

Although a series of specific of epidemiological data, such as isolation levels, amount of infectious and susceptible individuals, relative infectiousness and susceptibility, of said each and every risk group on said given period of time, and local population are gathered and stored, the computer-implemented method disclosed herein, may gather and store other epidemiological data for disease damage payments calculations. Said other epidemiological data, as used herein, means any other data different than the mentioned above suitable for calculating disease damage payments made by the spreaders fund, for example serological data from antibody prevalence studies like the published in (5).

Although a period of time of one day is a convenient time period for the purpose of the present invention, another period of time different than one day may be agreed to calculate the disease damage payments. Suitable period of time for the purposes of the present invention may be weeks as it is the preferred period of time used in epidemiology, but it can also be days, months, years or others.

In addition to the above, there may be more specific variants of the said computer-implemented method, which do not change the essentials, but nevertheless offer an interesting range of possibilities that help to customize the method according to the conditions of the environment in which it is applied, understanding by context both the conditions of the environment and the needs of the various parties involved, i.e. individuals and insurers, the corresponding groups, governments, and the like.

For example, in one aspect, the computer-implemented method may contemplate that said each insurer uses an agreed calculation method and its own epidemiological and actuarial models to estimate absolute value of future disease damage payments and hence determine its own IEIP premiums, wherein agreed calculation method, as used herein means any method that insurers (or government or other entities) agree to use for calculating disease damage payments, most likely before infections and disease damages occur; own epidemiological and actuarial models for an insurer, as used herein means any models used by an insurer to estimate the actual payments it will give and receive in the future given the agreed calculation method, its insured, the disease damages of its insured and other insured, and the evolution of the epidemic; and own IEIP premiums, as used herein means the amount of money the insured should charge to its insured for the IEIP .

In another aspect, the computer-implemented method may contemplate that the disease damage payments are made only to other IEIP holders; wherein the other IEIP holders, as used herein, means individuals that have purchased IEIP.

In another aspect, the computer-implemented method may contemplate that a key personnel government granted exemptions and some individuals unable to pay the IEIP are assumed to belong to different RG and their disease damage payments due to the spreader’s fault are covered by the government.

In another aspect, the computer-implemented method may contemplate that the disease damages are reported by another entity than the insurer of the injured individual, wherein said another entity reporting the disease damages, as used herein means any other entity different than an insurer, for example a government run health system.

In another aspect, the computer-implemented method may contemplate that entering hospitals or nursing homes, or not doing it with proper safety measures like hazmat suits, is not covered by IEIPs. The proper safety measures related with the target disease might be for example entering nursing homes with a recent negative PCR test.

In another aspect, the computer-implemented method may contemplate those individuals that have been infected, vaccinated, have antibodies or other immune responses, like T-cell reactivity, or any other immunizing event are removed from their original risk group.

In other aspects, the computer-implemented method may contemplate that the moment of disease damage occurrence or claims have a limit in time after infection/diagnosis and/or in the amount claimed. As an example, the limit in time after infection/diagnosis may range months or years. And the limit in the amount claimed, as used herein means a cap on the disease damage claim, for example 120.000 pound sterling (as it happens in the UK with the Vaccine Damage Payment).

In another aspect, the computer-implemented method may contemplate that the disease damage claims are tabulated; wherein the tabulation, as used herein, means that the amount of money that can be claimed is pre-established for the different disease damages.

In other aspects, the computer-implemented method may contemplate that some individuals are spared of purchasing the IEIP, and/or claiming damages. Namely, the reasons for be spared of purchasing the IEIP, and/or claiming damages are any reason that governments might deems appropriate, for example individuals not being reachable or not having means to purchase de IEIP, the fault as spreaders and/or the disease damages caused being so low to or by an RG that IEIP price is negligible or uneconomic to collect.

In another aspect, the computer-implemented method may contemplate that only one single insurer offers the IEIP.

In other aspects, the computer-implemented method may contemplate that a portion of premiums are charged/charged back to the spreaders if the disease damages end up being higher/lower than estimated, the above meaning that for example if the purchase price of the IEIP end up being too high because the estimated disease damages were overestimated then some of the excess money could be charged back to the insurers.

In another aspect, the computer-implemented method may contemplate that wherein the disease damage claims paid to the injured by their insurer might exceed tabulated ones.

In another aspect, the computer-implemented method may contemplate that the disease damage claims paid to injured by their insurer might differ from tabulated ones.

In other aspects, the computer-implemented method may contemplate those intermediate entities, including restaurants or concert halls, are allowed to buy IEIP on behalf of their customers; wherein the intermediate entities, as used herein means any entity being able to purchase IEIP on behalf of individuals being exempt of isolations, for example some rock concert buying IEIP for individuals attending that night.

In another aspect, the computer-implemented method may contemplate those exemptions and/or the disease damage payments are associated with other kinds of insurance, or not associated with any insurance at all.

In another aspect, the computer-implemented method may contemplate that the infected individuals are symptomatic (e.g. Ebola patient), or they already know that they are infectious (e.g. Typhoid Mary).

In another aspect, the computer-implemented method may contemplate that the disease damage claims might include other than disease related damages.

In another aspect, the computer-implemented method may contemplate that the disease damage claims might be corrected for individuals suffering them for other causes while being infected “with” the virus by chance.

In another aspect, the computer-implemented method may contemplate that said relative infectiousness and susceptibility in one location might be considerably lower due to prior immunity or change over time due to seasonality.

Furthermore, in another aspect, the computer-implemented method may contemplate that an epidemiological model with competing variants is used to calculate the disease damage payments.

### Exemplary Payment Calculation Method

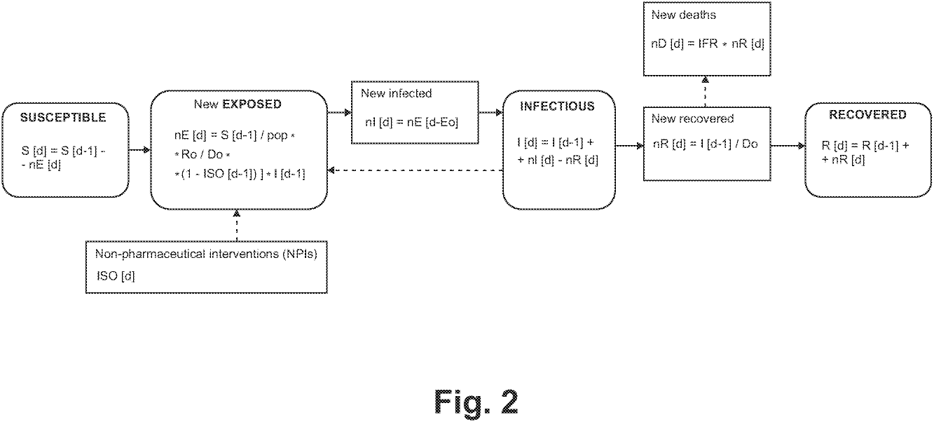

A shared fault calculation based on an N-stratum SEIR (Susceptible -> Exposed -> Infectious -> Recovered) epidemiological model is shown in FIG. 2.

In the SEIR model, individuals belong to either one of four compartments, new individuals Infectious in day d+Eo (daily period) are:

$$n I \left[d + E o\right] = \frac{S \left[d - 1\right]}{p o p} ∗ \frac{R o}{D o} ∗ \left(1 - I S O \left[d - 1\right]\right) ∗ I \left[d - 1\right]$$

Where:
Eo: average exposed interval (days)
S: remaining amount of susceptible individuals
pop: population (number of individuals in the location)
Ro: reproduction number (number of exposed per infectious at time 0 without NPIs) Do: average infectiousness interval (days)
ISO: average isolation contacts between individuals
I: amount of infectious individuals

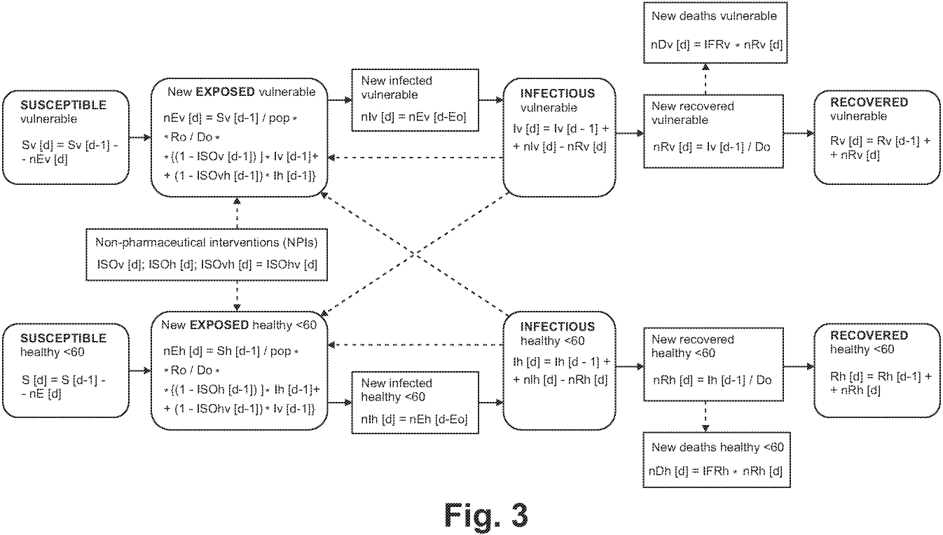

Since the objective is to allow different isolation levels in different risk groups, the SEIR model must be extended. Exemplary formulae deduction follows, as shown in FIG. 3, using the 2-stratum model of (1) and then is generalized to N-stratum.

New vulnerable individuals Infected in period d+Eo are:

$$\begin{matrix}\text{nIv} \left[\text{d} \text{+} \text{Eo}\right] = \frac{\text{Sv} \left[\text{d} - 1\right]}{\text{pop}} ∗ \frac{\text{Ro}}{\text{Do}} ∗ \text{Uv} \\ ∗ \left\{\left(1 - \text{ISOvv} \left[\text{d} - 1\right]\right) ∗ \text{Fv} ∗ \text{Iv} \left[\text{d} - 1\right] + \left(1 - \text{ISOvh} \left[\text{d} - 1\right]\right) ∗ \text{Fh} ∗ \text{Ih} \left[\text{d} - 1\right]\right\}\end{matrix}$$

Where:
Sv: remaining amount of susceptible individuals of the vulnerable stratum
Uv: average relative susceptibility of vulnerable individuals
ISOvv: average isolation contact between vulnerable individuals
Fv: average relative infectiousness of vulnerable individuals
Iv: number of infectious individuals of the vulnerable stratum
ISOvh: average joint isolation contact between vulnerable individuals and healthy <60 individuals
Fh: average relative infectiousness of healthy <60 individuals
Ih: number of infectious individuals of healthy <60 stratum

Vulnerable infected individual on period d+Eo will suffer in future periods uncertain disease damages related to the infection (i.e. death or squeals)

$$\text{Dv} \left[\text{d} \text{+} \text{Eo}\right]$$

and the proportion of those disease damages that correspond to the interaction with healthy <60 individuals is

$$\frac{\left(1 - I S O v h \left[d - 1\right]\right) ∗ F h ∗ I h \left[d - 1\right]}{\left(1 - I S O v v \left[d - 1\right]\right) ∗ F v ∗ I v \left[\text{d} - 1\right] + \left(1 - I S O v h \left[d - 1\right]\right) ∗ F h ∗ I h \left[d - 1\right]}$$

Beware that {3} is only the proportion of damage suffered by injured vulnerable in which they have shared fault with spreaders healthy <60 (it does not include the shared fault of injured vulnerable with vulnerable spreaders), where the sum of those proportions (V for vulnerable injured and H for spreaders healthy >60) is

$$\text{V} \text{+} \text{H} \text{=} 1$$

If all fault is assigned to Susceptible (H=0), and if all fault is assigned to Infectious (V=0). A more general approach is to assume that the second term in {2} depict faults that are: virus related (Ro/Do), joint interaction related (ISOvh), susceptible related (Sv/pop*Uv), infectious related (Ih*Fh), and that proportions V and H must also satisfy

$$\frac{\text{V}}{\text{Uv} ∗ \frac{\text{Sv} \left[\text{d} - \text{1}\right]}{\text{Pop}}} = \frac{\text{H}}{\text{Fh}}$$

and thus

$$\text{H=} \frac{\text{Fh}}{\text{Uv} ∗ \frac{\text{Sv} \left[d - 1\right]}{\text{Pop}} + \text{Fh}}$$

And particularly if ISOvh is assumed to satisfy

$$\left(1 - \text{ISOvh} \left[d - 1\right]\right) = {\left(1 - \text{ISOvv} \left[d - 1\right]\right)}^{\frac{1}{2}} ∗ {\left(1 - \text{ISOhh} \left[d - 1\right]\right)}^{\frac{1}{2}}$$

then

$$\begin{matrix}H = \\ \frac{F h ∗ {\left(1 - I S O h h \left[d - 1\right]\right)}^{\frac{1}{2}}}{U v ∗ \frac{S v \left[d - 1\right]}{P o p} ∗ {\left(1 - I S O v v \left[d - 1\right]\right)}^{\frac{1}{2}} + F h ∗ {\left(1 - I S O h h \left[d - 1\right]\right)}^{\frac{1}{2}}}\end{matrix}$$

Combining (3) and (8) the amount of disease damages suffered by vulnerable due to an infection in period d+Eo that is fault of spreaders healthy <60 is

$$\begin{matrix}{\text{D}}_{\text{v}}^{\text{h}} \left[\text{d+Eo}\right] = \frac{\text{Dv} \left[\text{d+Eo}\right]}{1 + \frac{{\left(1 - I S O v v \left[d - 1\right]\right)}^{\frac{1}{2}} ∗ F v ∗ I v \left[d - 1\right]}{{\left(1 - I S O h h \left[d - 1\right]\right)}^{\frac{1}{2}} ∗ F h ∗ I h \left[d - 1\right]}} \\ \text{} \text{} ∗ \frac{1}{1 + \frac{S v \left[d - 1\right] ∗ U v ∗ {\left(1 - I S O v v \left[d - 1\right]\right)}^{\frac{1}{2}}}{P o p ∗ F h ∗ {\left(1 - I S O h h \left[d - 1\right]\right)}^{\frac{1}{2}}}}\end{matrix}$$

And more generally, if there are N groups, for any given pair of groups j and i (including j=i), the amount of disease damages suffered by RG j due to an infection in period d+Eo that is fault of the spreaders of RG i is

$$\begin{matrix}{\text{D}}_{\text{j}}^{\text{i}} \left[\text{d} + \text{Eo}\right] = \\ \frac{\text{Dj} \left[\text{d} + \text{Eo}\right]}{{\sum }_{\text{k=1}}^{\text{N}} \left\{\frac{{\left(1 - I S O k k \left[d - 1\right]\right)}^{\frac{1}{2}} ∗ F k ∗ I k \left[d - 1\right]}{{\left(1 - I S O i i \left[d - 1\right]\right)}^{\frac{1}{2}} ∗ F i ∗ I i \left[d - 1\right]}\right\}} \\ ∗ \frac{1}{1 + \frac{S j \left[d - 1\right] ∗ U j ∗ {\left(1 - I S O j j \left[d - 1\right]\right)}^{\frac{1}{2}}}{P o p ∗ F i ∗ {\left(1 - I S O i i \left[d - 1\right]\right)}^{\frac{1}{2}}}}\end{matrix}$$

Damages fault of spreaders paid by all N RGs to injured of RG j is

$${\sum }_{i = 1}^{N} {\text{D}}_{\text{j}}^{\text{i}} \left[\text{d} + \text{Eo}\right]$$

Damages fault of spreaders paid by RG i to injured of all N RGs is

$${\sum }_{j = 1}^{N} {\text{D}}_{\text{j}}^{\text{i}} \left[\text{d} + \text{Eo}\right]$$

Each insurer receives and pays an amount proportional to the number insured (i.e. susceptible in each RG) and injured that it has of each RG.

Damages paid due to each spreader of RG i is

$$\frac{{\sum }_{j = 1}^{N} {\text{D}}_{\text{j}}^{\text{i}} \left[\text{d} + \text{Eo}\right]}{{S}_{i}}$$

where Si: remaining susceptible of RG i.

### Exemplary Results for Real Sars-Cov-2 Epidemic

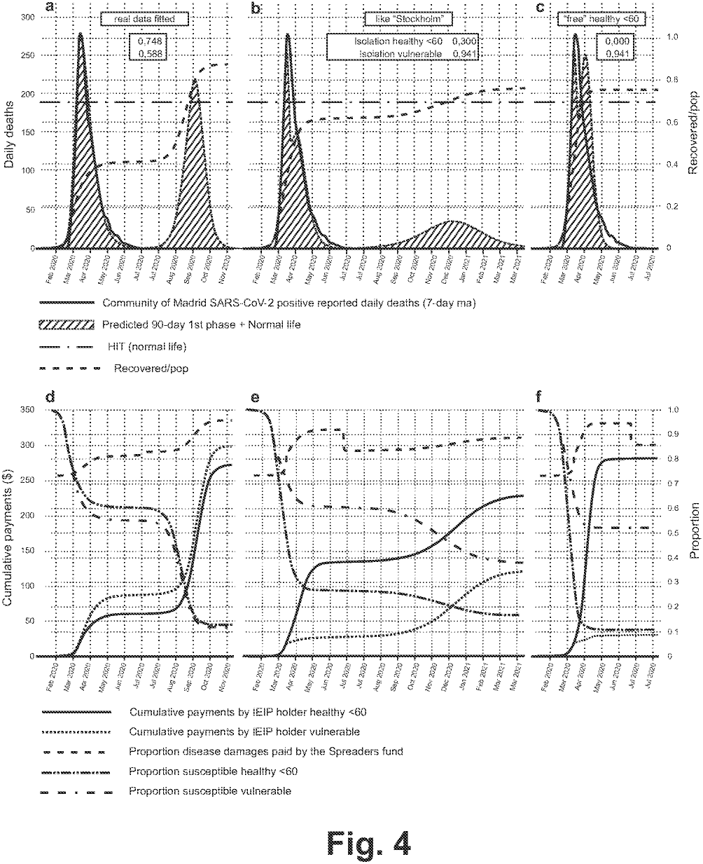

Exemplary calculation results using the method with formula {9} implemented in (14) for the Community of Madrid are shown in FIG. 4.

Individuals are grouped in two RG: healthy <60, and vulnerable individuals (i.e. >60 years old and <60 with comorbidities). FIG. 4.a shows inferred isolations in each RG (i.e. 0.748 for healthy <60 and 0.588 to vulnerable) needed to fit reported daily deaths and age serology ratio in Madrid during the first wave between March and June 2020. FIG. 4.b shows estimated daily deaths if isolations applied in Madrid would have been inferred for Stockholm during the first wave (i.e. 0.300 for healthy <60 and 0.941 to vulnerable). FIG. 4.c shows estimated daily deaths if isolations applied in Madrid would have been 0.941 for vulnerable and 0.00 to healthy <60 (i.e. no isolations).

Disease damages are assumed to be USD 50.000 for each vulnerable death and USD 250.000 for each healthy <60 death. Damages are assigned to infections that occurred 18 days before.

FIG. 4.d shows the cumulative payments made by the spreaders fund per each IEIP holder (e.g. $53.78 per healthy <60 and $75.54 per vulnerable up to Apr. 25, 2021) under the “real data fitted” scenario. The amount of susceptible that belong to each RG are a diminishing fraction of the initials due to infections (e.g. 62% of initial for healthy <60, and 56% for vulnerable on Apr. 25, 2021).

If healthy <60 individuals DO NOT ISOLATE AT ALL (FIG. 4.f), for each one that remains susceptible (i.e. not infected) until Herd Immunity is reached, the Spreaders fund ends up paying $280 (two hundred and eighty US dollars).

And if a focused protection strategy “like Stockholm” is achieved (FIG. 4.d), those payments are $230 from March 2020 to February 2021. Only $19 per month to avoid Madrid’s mandatory isolations while remaining susceptible (i.e. not vaccinated and not vaccinated).

Notice that in all three scenarios in every moment the proportion of disease damages that is paid by the Spreaders fund is higher than 70% (dashed line).

### Additional Considerations

FIG. 4 results use isolation levels Ihh and Ivv estimated using the model (1) to fit age serology ratio (e.g. 0.79 in Madrid and 1.71 in Stockholm) assuming that the joint isolation ISOvh is equal to ISOhv and that formula {7} holds.

A more general approach might include estimating ISOij (ij element in the contact matrix) using proximity with a mobile app, like “Exposure Notifications” from Google and Apple (7), and instead of using {8} to calculate the proportion of fault in the joint interaction, discriminate the right of each RG to be in each place where interactions happen (e.g. children not isolating from elderly in a primary schools might not have the same fault share that in restaurants).

Parameters U and F might be estimated worldwide and used in local calculations, and can vary over time due seasonality or new variants. For example, youngster might have lower infectiousness F because they tend to have a higher asymptomatic proportion (5), individuals that keep asymptomatic are rarely contagious (8), and children have lower susceptibility (5), vaccinated might have lower susceptibility than unvaccinated (11) (although VE might wane with time), and recovered from infections have even lower susceptibility than vaccinated (12).

Infections I in each RG can be estimated using serological data (5) and corrected downward using the parameter U for each RG. Or using symptomatic PCR+ and corrected upward estimating the proportion of undetected cases in each RG.

As depicted in (2) other scenarios might also be included into the SEIR model: vaccination, Ro, Eo, and Do variations due to seasonality, the emergence of new competing variants, or the occurrence of deaths for other causes while testing positive for the virus.

### REFERENCES

1. Levan Djaparidze, 2-stratum SEIRS model, www.sars2seir.com/paper-10-2020/

2. Levan Djaparidze and Federico Lois, SARS-CoV-2 waves in Europe: A 2-stratum SEIRS model solution, https://www.medrxiv.org/content/10.1101/2020.10.09.20210146v3.

3. Hao-Yuan Cheng et al, Contact Tracing Assessment of COVID-19 Transmission Dynamics in Taiwan and Risk at Different Exposure Periods Before and After Symptom Onset, https://jamanetwork.com/journals/jamainternalmedicine/fullarticle/2765641.

4. Roman Wölfel et al, Virological assessment of hospitalized patients with COVID-2019, https://www.nature.com/articles/s41586-020-2196-x

5. Estudio Nacional de sero-Epidemiología de la infección por SARS-CoV-2 en España (ENECOVID), https://portalcne.isciii.es/enecovid19/

6. https://www.statista.com/statistics/1191568/reported-deaths-from-covid-by-age-us/

7. https://www.google.com/covid19/exposurenotifications/

8. Shiyi Cao et al, Post-lockdown SARS-CoV-2 nucleic acid screening in nearly ten million residents of Wuhan, China, https://www.nature.com/articles/s41467-020-19802-w

9. Leif Agerhom Roll, U.S. Pat. 10,949,928 B

10. COVID-19 savages U.S. economy, 2020 performance worst in 74 years, https://www.reuters.com/article/us-usa-economy-idUSKBN29X0I8

11. Hiam Chemaitelly et al, Waning of BNT162b2 vaccine protection against SARS-CoV-2 infection in Qatar, https://www.medrxiv.org/content/10.1101/2021.08.25.21262584v1.full

12. Sivan Gazit et al, Comparing SARS-CoV-2 natural immunity to vaccine-induced immunity: reinfections versus breakthrough infections, https://www.medrxiv.org/content/10.1101/2021.08.24.21262415v1

13. NYC’s Covid-19 Vaccination Proof Mandate for Restaurants and Dining: What to Know, https://www.wsj.com/articles/nycs-vaccination-proof-mandate-for-restaurants-and-dining-what-to-know-11631540546

14. Levan Djaparidze, EXEMPLARY SYSTEM AND METHOD TO GATHER AND STORE EPIDEMIOLOGICAL DATA TO CALCULATE DAMAGE PAYMENTS OF AN INFECTIOUS DISEASE SPREADERS FUND, www.sars2seir.com/patent/

15. Pandemic Risk Protection Accelerate Recovery and Build Resilience Now Through Public-Private Partnership, June 2020, https://info.marsh.com/I/395202/2020-06-01/bmbxvj/395202/207425/pandemic_risk_protection_report.pdf

16. Neil Ferguson et al, Report 49: Growth, population distribution and immune escape of Omicron in England, https://www.imperial.ac.uk/media/imperial-college/medicine/mrc-gida/2021-12-16-COVID19-Report-49.pdf

17. Explained: Austria’s new law that makes Covid-19 vaccination mandatory, https://indianexpress.com/article/explained/explained-austria-new-law-covid-19-vaccination-mandatory-7757973/

18. Mandatory Vaccination and the Failure of Modern Constitutional Law, https://libertarianinstitute.org/articles/mandatory-vaccination-and-the-failure-of-modern-constitutional-law/

What is claimed is:

1. A computer-implemented method for calculating disease damage payments due to spreaders fault associated to an isolation exemption insurance policy (IEIP), the computer-implemented method comprising the followings steps:
a) gathering and storing disease damage claims of individuals of each and every risk group infected on a given period of time,
b) gathering and storing epidemiological data, such as isolation levels, amount of infectious and susceptible individuals, relative infectiousness and susceptibility, of said each and every risk group on said given period of time, and local population,
c) calculating disease damages suffered by said each and every risk group due to an infection on said given period of time, that were fault of spreaders of said each and every risk group, and
d) calculating the disease damage payments that each insurer must make to, or receive from, a spreaders fund for each said insurer.

2. The computer-implemented method of claim 1, wherein other formulas and/or steps are agreed to calculate the disease damage payments.

3. The computer-implemented method of claim 1, wherein other epidemiological data is gathered and stored for disease damage payments calculations.

4. The computer-implemented method of claim 1, wherein other periods of time different than one day are agreed to calculate the disease damage payments.

5. The computer-implemented method of claim 1, wherein said each insurer uses an agreed calculation method and its own epidemiological and actuarial models to estimate absolute value of future disease damage payments and hence determine its own IEIP premiums.

6. The computer-implemented method of claim 1, wherein the disease damage payments are made only to other IEIP holders.

7. The computer-implemented method of claim 1, wherein a key personnel government granted exemptions and some individuals unable to pay the IEIP are assumed to belong to different RG and their disease damage payments due to the spreaders fault are covered by the government.

8. The computer-implemented method of claim 1, wherein the disease damages are reported by another entity than the insurer of the injured individual.

9. The computer-implemented method of claim 1, wherein entering hospitals or nursing homes, or not doing it with proper safety measures like hazmat suits, is not covered by IEIPs.

10. The computer-implemented method of claim 1, wherein individuals that have been infected, vaccinated, have antibodies, or other immune response, like T-cell reactivity, or any other immunizing event are removed from their original risk group.

11. The computer-implemented method of claim 1, wherein the moment of disease damage occurrence or claims have a limit in time after infection/diagnosis and/or in the amount claimed.

12. The computer-implemented method of claim 1, wherein the disease damage claims are tabulated.

13. The computer-implemented method of claim 1, wherein only one single insurer offers the IEIP.

14. The computer-implemented method of claim 1, wherein a portion of premiums are charged/charged back to the spreaders if the disease damages end up being higher/lower than estimated.

15. The computer-implemented method of claim 1, wherein intermediate entities, including restaurants or concert halls, are allowed to buy IEIP on behalf of their customers.

16. The computer-implemented method of claim 1, wherein exemptions and/or the disease damage payments are associated with other kinds of insurance, or not associated with any insurance at all.

17. The computer-implemented method of claim 1, wherein the disease damage claims might be corrected for individuals suffering them for other causes while being infected with the virus by chance.

18. The computer-implemented method of claim 1, wherein said relative infectiousness and susceptibility in one location might be considerably lower due to prior immunity or change over time due to seasonality.

19. The computer-implemented method of claim 1, wherein epidemiological model with competing variants is used to calculate the disease damage payments.
